SEQUENCE LISTING (4 sequences)

SEQ ID NO: 1 - RNA, 22 nt, Homo sapiens
uaugugccuuuggacuacaucg

SEQ ID NO: 2 - RNA, 21 nt, Homo sapiens
gcaguccaugggcauauacac

SEQ ID NO: 3 - RNA, 22 nt, Homo sapiens
agccccugcccaccgcacacug

SEQ ID NO: 4 - RNA, 22 nt, Homo sapiens
cugugcgugugacagcggcuga

(19) United States
(12) Patent Application Publication
(10) Pub. No.: US 2016/0369343 A1
(43) Pub. Date: Dec. 22, 2016

(54) PREDICTION OF PREECLAMPSIA USING MICRORNA

(71) Applicants: FRIEDRICH MIESCHER INSTITUTE FOR BIOMEDICAL RESEARCH, Basel (CH); UNIVERSITY HOSPITAL BASEL, Binningen (CH)

(72) Inventors: Marc BUEHLER, Riehen (CH); Sebastien LALEVEE, Montpellier (FR); Olav LAPAIRE, Binningen (CH)

(21) Appl. No.: 15/122,177

(22) PCT Filed: Feb. 26, 2015
(86) PCT No.: PCT/IB2015/051442

§ 371 Date: Aug. 28, 2016

(30) Foreign Application Priority Data
Feb. 28, 2014 (EP) 14157298.2

(51) Int. Cl. C12Q 1/68; G01N 33/68

(52) U.S. Cl. CPC C12Q 1/6883; G01N 33/689; C12Q 2600/178; C12Q 2600/158; G01N 2800/368; G01N 2800/50

(57) ABSTRACT

The application relates to a method for predicting a risk for preeclampsia in a subject, said method comprising analysing a sample from the subject for the level of expression of miR455 and comparing the level of expression of miR455 in the sample from the subject to the levels of miR455 in a control sample, wherein a significantly lower expression of miR455 as compared to the expression of miR455 in the control is indicative of a risk for preeclampsia.

The placenta is the organ that connects the developing foetus to the uterine wall to allow gas exchange, nutrient uptake, and elimination of waste products via the mother's blood supply. Moreover, the placenta has an endocrine function and produces various pregnancy-associated hormones and growth factors to regulate fetal growth and the maternal response to the pregnancy (Murphy et al., Endocr Rev. 2006 Apr.; 27(2):141-69). Aberrant function or development of the placenta has been associated with many pregnancy complications, such as preeclampsia (PE). PE is a multisystemic, pregnancy-associated disorder, with an incidence of 2-5% and represents a major cause of maternal and fetal morbidity and mortality (Steegers et al. Lancet. 2010 Aug. 21;376(9741):631-44). Although the exact aetiology of PE remains elusive, the placenta plays a central role. In the first and second trimester, local aberrant feto-maternal immune interactions within the uterine wall lead to impaired arterial wall invasion by trophoblast cells. This results in failed transformation of the uterine spiral arteries and subsequently decreased placental perfusion. Chronic hypoxia or alternate periods of hypoxia/re-oxygenation within the intervillous space triggers tissue oxidative stress and increase placental apoptosis and necrosis (Roberts et al. Placenta. 2009 Mar.; 30 Suppl A:S32-7). Subsequently, placental debris and the aberrant expression of pro-inflammatory, anti-angiogenic and angiogenic factors, lead to a systemic endothelial cell dysfunction and an exaggerated inflammatory response (Verlohren, S., H. Stepan, et al. (2012). “Angiogenic growth factors in the diagnosis and prediction of pre-eclampsia.” Clinical science 122(2): 43-52; Young, B. C., R. J. Levine, et al. (2010). “Pathogenesis of preeclampsia.” Annual review of pathology 5: 173-192; Redman, C. W. (2011). “Preeclampsia: a multi-stress disorder.” La Revue de medecine interne/fondee . . . par la Societe nationale francaise de medecine interne 32 Suppl 1: S41-44; Wang, A., S. Rana, et al. (2009). “Preeclampsia: the role of angiogenic factors in its pathogenesis.” Physiology 24: 147-158; George, E. M. and J. P. Granger (2010). “Recent insights into the pathophysiology of preeclampsia.” Expert review of obstetrics & gynecology 5(5): 557-566).

MicroRNAs (miRNAs) are a large family of post-transcriptional regulators of gene expression that are about 21 nucleotides in length and control many developmental and cellular processes in eukaryotic organisms. miRNAs are processed from precursor molecules (pri-miRNAs), which are either transcribed from independent miRNA genes or represent introns of protein coding genes. pri-miRNAs fold into hairpins that are sequentially processed by the nuclear RNAse III enzyme Drosha into roughly 70-nucleotide pre-miRNAs. After export to the cytoplasm, the pre-miRNA gets further processed by Dicer to a 21-bp miRNA/miRNA* duplex. One strand of this duplex, representing a mature miRNA, is then incorporated into the miRNA-induced silencing complex (miRISC) (Krol J et al, Nat Rev Genet. 2010 Sep.; 11(9):597-610). Most miRNA genes produce one dominant mature miRNA species, from either the 5′ or 3′ arm of the pre-miRNA hairpin, which is preferentially incorporated into miRISC. However, some miRNA genes yield mature miRNAs from both arms, which are annotated using -5p and −3p suffixes (Chiang, H. R., L. W. Schoenfeld, et al. (2010). “Mammalian microRNAs: experimental evaluation of novel and previously annotated genes.” Genes & development 24(10): 992-1009; Griffiths-Jones, S. (2004). “The microRNA Registry.” Nucleic acids research 32(Database issue): D109-111).

As part of miRISC, mature miRNAs base pair to target mRNAs and direct their translational repression, mRNA deadenylation and degradation, or a combination of the two (Krol J et al, Nat Rev Genet. 2010 Sep.; 11(9):597-610). Most animal miRNAs imperfectly base pair with sequences in the 3′-UTR of target mRNAs. However, efficient mRNA targeting requires continuous base pairing of the miRNA “seed” sequence (nucleotides 2 to 8) (Bartel D P. Cell. 2009 Jan. 23;136(2):215-33). Because more extensive complementarity than seed pairing is unusual in animals, predicting miRNA target mRNAs computationally has remained a challenge. Nonetheless, several computational tools for predicting potential miRNA targets haven been developed (Bartel D P. Cell. 2009 Jan. 23;136(2):215-33).

Profiling of miRNA expression has revealed that some miRNAs are expressed universally, whereas others are expressed tissue specifically (Liang, Y., D. Ridzon, et al. (2007). “Characterization of microRNA expression profiles in normal human tissues.” BMC genomics 8: 166), and accumulating evidence shows that miRNAs are frequently deregulated in human malignancies and can function as oncogenes or tumor suppressor genes (Ventura, A. and T. Jacks (2009). “MicroRNAs and cancer: short RNAs go a long way.” Cell 136(4): 586-591; Hamilton, M. P., M. O. Gore, et al. (2011). “Adiponectin and cardiovascular risk profile in patients with type 2 diabetes mellitus: parameters associated with adiponectin complex distribution.” Diabetes & vascular disease research: official journal of the International Society of Diabetes and Vascular Disease 8(3): 190-194). In the human placenta, two large clusters of microRNA genes are encoded on chromosome 14 (C14MC) and chromosome 19 (C19MC) (Morales-Prieto, D. M., W. Chaiwangyen, et al. (2012). “MicroRNA expression profiles of trophoblastic cells.” Placenta 33(9): 725-734; Girardot, M., J. Cavaille, et al. (2012). “Small regulatory RNAs controlled by genomic imprinting and their contribution to human disease.” Epigenetics: official journal of the DNA Methylation Society 7(12): 1341-1348). Interestingly, expression of certain placenta specific microRNAs is deregulated in cancer tissues, although the functional role of these miRNAs has remained elusive (Girardot, M., J. Cavaille, et al. (2012). “Small regulatory RNAs controlled by genomic imprinting and their contribution to human disease.” Epigenetics: official journal of the DNA Methylation Society 7(12): 1341-1348; Louwen, F., C. Muschol-Steinmetz, et al. (2012). “A lesson for cancer research: placental microarray gene analysis in preeclampsia.” Oncotarget 3(8): 759-773). Few placental-specific microRNAs have been associated with placental disorders such as PE (Doridot, L., F. Miralles, et al. (2013). “Trophoblasts, invasion, and microRNA.” Frontiers in genetics 4: 248; Morales-Prieto, D. M., W. Chaiwangyen, et al. (2012). “MicroRNA expression profiles of trophoblastic cells.” Placenta 33(9): 725-734). For example, several studies revealed an upregulation of the miRNA miR210 in placenta from PE patients (Pineles, B. L., R. Romero, et al. (2007). “Distinct subsets of microRNAs are expressed differentially in the human placentas of patients with preeclampsia.” American journal of obstetrics and gynecology 196(3): 261 e261-266; Mayor-Lynn, K., T. Toloubeydokhti, et al. (2011). “Expression Profile of MicroRNAs and mRNAs in Human Placentas From Pregnancies Complicated by Preeclampsia and Preterm Labor.” Reproductive sciences 18(1): 46-56; Enquobahrie, D. A., D. F. Abetew, et al. (2011). “Placental microRNA expression in pregnancies complicated by preeclampsia.” American journal of obstetrics and gynecology 204(2): 178 e112-121; Zhu, X. M., T. Han, et al. (2009). “Differential expression profile of microRNAs in human placentas from preeclamptic pregnancies vs normal pregnancies.” American journal of obstetrics and gynecology 200(6): 661 e661-667; Ishibashi, O., A. Ohkuchi, et al. (2012). “Hydroxysteroid (17-beta) dehydrogenase 1 is dysregulated by miR-210 and miR-518c that are aberrantly expressed in preeclamptic placentas: a novel marker for predicting preeclampsia.” Hypertension 59(2): 265-273). However, most of those studies have been limited by the number of placental samples used for microRNA expression, their heterogeneity and/or the limited number of microRNA studied (Zhu, X. M., T. Han, et al. (2009). “Differential expression profile of microRNAs in human placentas from preeclamptic pregnancies vs normal pregnancies.” American journal of obstetrics and gynecology 200(6): 661 e661-667; Mayor-Lynn, K., T. Toloubeydokhti, et al. (2011). “Expression Profile of MicroRNAs and mRNAs in Human Placentas From Pregnancies Complicated by Preeclampsia and Preterm Labor.” Reproductive sciences 18(1): 46-56). Thus, there is a need in the art for miRNAs different from miR210 which would be differentially expressed in PE patients and could allow a diagnostic method.

The present inventors realised that in order to respond to this need, it would be best to concentrate on particular trophoblast cells.

Trophoblast cells are the specialized cells of the placenta that play an important role in embryo implantation and interaction with the maternal uterus. Two different trophoblast differentiation pathways occur during placental development. In the extravillous pathway, the cells either differentiate into interstitial extravillous trophoblasts that invade the decidua and a part of the myometrium, or endovascular extravillous trophoblasts that remodel the maternal vessels. In the villous pathway, cytotrophoblast cells fuse to a multinucleated syncytiotrophoblast layer that covers the entire surface of the placenta (Ji, L., J. Brkic, et al. (2013). “Placental trophoblast cell differentiation: Physiological regulation and pathological relevance to preeclampsia.” Molecular aspects of medicine 34(5): 981-1023). This syncytium is in direct contact with the maternal blood and thus facilitates the exchange of nutrients, wastes and gases between the maternal and fetal systems. Defective cytotrophoblast to syncytiotrophoblast differentiation has been proposed to be involved in the etiology of PE (Huppertz et al. Hypertension. 2008 Apr;51(4):970-5).

However, because the placenta is a complex and heterogeneous organ, detailed molecular study of mechanisms underlying placental biology are very challenging if not impossible. Therefore, the use of cellular models is desirable. To study microRNAs during villous trophoblast cell differentiation, the present inventors took advantage of an established cytotrophoblast-like (CT) cell line (BeWo). Using next-generation small RNA sequencing, the inventors' analysis revealed two related miRNAs (miR455-5P/-3P) that were reproducibly upregulated upon cyto- to syncytiotrophoblast differentiation. Target prediction and validation experiments showed that miR455-3P restrains a hypoxia response that otherwise could prevent cytotrophoblast to syncytiotrophoblast differentiation. Importantly, they found that expression of miR455 was significantly downregulated in 15 investigated PE cases compared to 14 healthy donor controls, whereas the levels of other placenta-specific miRNAs remained unaffected.

The present invention hence provides a method for predicting a risk for preeclampsia in a subject, said method comprising analysing a sample from the subject for the level of expression of miR455 and comparing the level of expression of miR455 in the sample from the subject to the levels of miR455 in a control sample, wherein a significantly lower expression of miR455 as compared to the expression of miR455 in the control is indicative of a risk for preeclampsia.

In some embodiments of the invention, the expression of miR210 is also analysed in the sample from the subject and wherein the combination of a significantly lower expression of miR455 and a significantly higher expression of miR210, as compared to the control sample, is indicative of a risk for preeclampsia. This combined use of two markers wherein one is overexpressed whereas the other one is underexpressed is particularly advantageous as it automatically provides an internal control for the assay.

In some embodiments, the ration miR210/miR455 is determined and a ratio greater than one is indicative of a risk for preeclampsia.

In some embodiments of the invention, the sample comprises placenta tissue.

In other embodiments of the invention, the sample is a blood sample.

In some embodiments, the subject is human.

The present invention also provides a kit for performing the method of the invention as described herein-above, said kit comprising means for analysing in a sample from a subject the level of expression of miR455; and, optionally, means for analysing in a sample from a subject the level of expression of miR210; and, optionally means for comparing the level of said miRNAs, i. e. miR455, and, optionally miR210, in a control sample.

The means can be a specific binding molecule, such as an oligonucleotide probe, antibody, or aptamer.

The present invention also encompasses the use of miR455 as a biomarker for preeclampsia, either alone or in combination with another biomarker, such as miR210.

Examples of suitable samples include biopsies, samples excised during surgical procedures, blood samples, urine samples, sputum samples, cerebrospinal fluid samples, and swabbed samples (such as saliva swab samples).

By “control sample” we mean a sample, equivalent to that from the subject, that has been derived from an individual that is not suffering from preeclampsia. Although equivalent tissue or organ samples, constituting control samples, or extracts from such samples, may be used directly as the source of information regarding levels of miRNA, in the control sample, it will be appreciated, and generally be preferred, that information regarding the expression of the levels of miRNA, in an “ideal” control sample be provided in the form of reference data. Such reference data may be provided in the form of tables indicative of the levels of miRNA in the chosen control tissue. Alternatively, the reference data may be supplied in the form of computer software containing retrievable information indicative of the levels of miRNA in the chosen control tissue. The reference data may, for example, be provided in the form of an algorithm enabling comparison of expression of at least the levels of miRNA, in the subject with expression of this miRNA in the control tissue sample.

In the event that the levels of miRNA in a control sample is to be investigated via processing of a tissue or organ sample constituting the control sample, it is beneficial that such processing is conducted using the same methods used to process the sample from the subject. Such parallel processing of subject samples and control samples allows a greater degree of confidence that comparisons of gene expression in these tissues will be normalised relative to one another (since any artefacts associated with the selected method by which tissue is processed and gene expression investigated will be applied to both the subject and control samples).

The method according to the invention will involve the analysis of at least the levels of miR-455. The finding that altered levels of miR-455 may be used in determining the effectiveness of a therapy.

The presence, absence and/or levels of miRNA may be detected using suitable probe molecules. Such detection will provide information as to the presence, absence and/or levels of miRNA and thereby allow comparison between the levels of miRNA occurring in the subject and those occurring in the control sample. Probes will generally be capable of binding specifically to the miRNA directly or indirectly. Binding of such probes may then be assessed and correlated with the levels of the miRNA to allow an effective prognostic comparison between the subject and the control.

By “altered expression” we include where the gene expression is either elevated or reduced in the sample when compared to the control, as discussed above.

Conversely by “unaltered expression” we include where the gene expression is not elevated or reduced in the sample when compared to the control, as discussed above.

An assessment of the levels of a miRNA and of whether a gene expression is altered or unaltered can be made using routine methods of statistical analysis.

It will be understood that “nucleic acids” or “nucleic acid molecules” for the purposes of the present invention refer to deoxyribonucleotide or ribonucleotide polymers in either single-or double-stranded form. Furthermore, unless the context requires otherwise, these terms should be taken to encompass known analogues of natural nucleotides that can function in a similar manner to naturally occurring nucleotides.

Furthermore it will be understood that target nucleic acids suitable for use in accordance with the invention need not comprise “full length” nucleic acids (e.g. full length gene transcripts), but need merely comprise a sufficient length to allow specific binding of probe molecules.

In an embodiment of the method of the invention, samples may be treated to isolate RNA target molecules by a process of lysing cells taken from a suitable sample (which may be achieved using a commercially available lysis buffer such as that produced by Qiagen Ltd.) followed by centrifugation of the lysate using a commercially available nucleic acid separation column (such as the RNeasy midi spin column produced by Qiagen Ltd). Other methods for RNA extraction include variations on the phenol and guanidine isothiocyanate method of Chomczynski, P. and Sacchi, N. (1987) Analytical Biochemistry 162, 156. “Single Step Method of RNA Isolation by Acid Guanidinium Thiocyanate-Phenol-Chloroform Extraction.” RNA obtained in this manner may constitute a suitable target molecule itself, or may serve as a template for the production of target molecules representative of the levels of miR-455

In the case of assessing the expression of chosen gene in order to have a more robust prediction value, it may be preferred that RNA derived from a subject or control sample may be used as substrate for cDNA synthesis, for example using the Superscript System (Invitrogen Corp.). The resulting cDNA may then be converted to biotinylated cRNA using the BioArray RNA Transcript labelling Kit (Enzo Life Sciences Inc.) and this cRNA purified from the reaction mixture using an RNeasy mini kit (Qiagen Ltd). mRNA, representative of gene expression, may be measured directly in a tissue derived from a subject or control sample, without the need for mRNA extraction or purification. For example, mRNA present in, and representative of gene expression in, a subject or control sample of interest may be investigated using appropriately fixed sections or biopsies of such a tissue. The use of samples of this kind may provide benefits in terms of the rapidity with which comparisons of expression can be made, as well as the relatively cheap and simple tissue processing that may be used to produce the sample. In situ hybridisation techniques represent preferred methods by which gene expression may be investigated and compared in tissue samples of this kind. Techniques for the processing of tissues of interest that maintain the availability of RNA representative of gene expression in the subject or control sample are well known to those of skill in the art. However, techniques by which mRNAs representative of gene expression in a subject or control sample may be extracted and collected are also well known to those skilled in the art, and the inventors have found that such techniques may be advantageously employed in accordance with the present invention. Samples comprising extracted mRNA from a subject or control sample may be preferred for use in the method of the third aspect of the invention, since such extracts tend to be more readily investigated than is the case for samples comprising the original tissues. For example, suitable target molecules allowing for comparison of gene expression may comprise the total RNA isolated from a sample of tissue from the subject, or a sample of control tissue. Furthermore, extracted RNA may be readily amplified to produce an enlarged mRNA sample capable of yielding increased information on gene expression in the subject or control sample. Suitable examples of techniques for the extraction and amplification of mRNA populations are well known, and are considered in more detail below.

By way of example, methods of isolation and purification of nucleic acids to produce nucleic acid targets suitable for use in accordance with the invention are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In the event that it is desired to amplify the nucleic acid targets prior to investigation and comparison of gene expression it may be preferred to use a method that maintains or controls for the relative frequencies of the amplified nucleic acids in the subject or control tissue from which the sample is derived.

Suitable methods of “quantitative” amplification are well known to those of skill in the art. One well known example, quantitative PCR, involves simultaneously co-amplifying a control sequence whose quantities are known to be unchanged between control and subject samples. This provides an internal standard that may be used to calibrate the PCR reaction.

In addition to the methods outlined above, the skilled person will appreciate that any technology coupling the amplification of gene-transcript specific product to the generation of a signal may also be suitable for quantitation. A preferred example employs convenient improvements to the polymerase chain reaction (U.S. Pat. Nos. 4,683,195 and 4,683,202) that have rendered it suitable for the exact quantitation of specific mRNA transcripts by incorporating an initial reverse transcription of mRNA to cDNA. Further key improvements enable the measurement of accumulating PCR products in real-time as the reaction progresses.

In many cases it may be preferred to assess the degree of gene expression in subject or control samples using probe molecules capable of indicating the presence of target molecules in the relevant sample.

Probes may be selected with reference to the product (direct or indirect) of gene expression to be investigated. Examples of suitable probes include oligonucleotide probes, antibodies, aptamers, and binding proteins or small molecules having suitable specificity.

Oligonucleotide probes can be used as probes. The generation of suitable oligonucleotide probes is well known to those skilled in the art (Oligonucleotide synthesis: Methods and Applications, Piet Herdewijn (ed) Humana Press (2004)). Oligonucleotide and modified oligonucleotides are commercially available from numerous companies.

For the purposes of the present description, an oligonucleotide probe may be taken to comprise an oligonucleotide capable of hybridising specifically to a nucleic acid target molecule of complementary sequence through one or more types of chemical bond. Such binding may usually occur through complementary base pairing, and usually through hydrogen bond formation. Suitable oligonucleotide probes may include natural (i.e., A, G, C, or T) or modified bases (7-deazaguanosine, inosine, etc.). In addition, a linkage other than a phosphodiester bond may be used to join the bases in the oligonucleotide probe(s), so long as this variation does not interfere with hybridisation of the oligonucleotide probe to its target. Thus, suitable oligonucleotide probes may be peptide nucleic acids in which the constituent bases are joined by peptide bonds rather than phosphodiester linkages.

As explained herein, microRNA molecules (“miRNAs”) are generally 21 to 22 nucleotides in length, though lengths of 17 and up to 25 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved by an enzyme called Dicer in animals. Dicer is ribonuclease III-like nuclease. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al, 1999; Seggerson et al, 2002). SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target (Denli et al, 2003).

The study of endogenous miRNA molecules is for instance described in U.S. Patent Application 60/575,743. Synthetic miRNAs are apparently active in the cell when the mature, single-stranded RNA is bound by a protein complex that regulates the translation of mRNAs that hybridize to the miRNA. Introducing exogenous RNA molecules that affect cells in the same way as endogenously expressed miRNAs requires that a single-stranded RNA molecule of the same sequence as the endogenous mature miRNA be taken up by the protein complex that facilitates translational control. A variety of RNA molecule designs have been evaluated. Three general designs that maximize uptake of the desired single-stranded miRNA by the miRNA pathway have been identified. An RNA molecule with a miRNA sequence having at least one of the three designs is referred to as a synthetic miRNA.

Synthetic miRNAs of the invention comprise, in some embodiments, two RNA molecules wherein one RNA is identical to a naturally occurring, mature miRNA. The RNA molecule that is identical to a mature miRNA is referred to as the active strand. The second RNA molecule, referred to as the complementary strand, is at least partially complementary to the active strand. The active and complementary strands are hybridized to create a double-stranded RNA, called the synthetic miRNA, that is similar to the naturally occurring miRNA precursor that is bound by the protein complex immediately prior to miRNA activation in the cell. Maximizing activity of the synthetic miRNA requires maximizing uptake of the active strand and minimizing uptake of the complementary strand by the miRNA protein complex that regulates gene expression at the level of translation. The molecular designs that provide optimal miRNA activity involve modifications to the complementary strand.

Two designs incorporate chemical modifications in the complementary strand. The first modification involves creating a complementary RNA with a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules including NH2, NHCOCH3, biotin, and others.

The second chemical modification strategy that significantly reduces uptake of the complementary strand by the miRNA pathway is incorporating nucleotides with sugar modifications in the first 2-6 nucleotides of the complementary strand. It should be noted that the sugar modifications consistent with the second design strategy can be coupled with 5′ terminal modifications consistent with the first design strategy to further enhance synthetic miRNA activities.

A third synthetic miRNA design involves incorporating nucleotides in the 3′ end of the complementary strand that are not complementary to the active strand.

Hybrids of the resulting active and complementary RNAs are very stable at the 3′ end of the active strand but relatively unstable at the 5′ end of the active strand. Studies with siRNAs indicate that 5′ hybrid stability is a key indicator of RNA uptake by the protein complex that supports RNA interference, which is at least related to the miRNA pathway in cells. The judicious use of mismatches in the complementary RNA strand significantly enhances the activity of the synthetic miRNA.

As explained herein, the term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self- complementary strand(s) or “complement(s)” of a particular sequence comprising a molecule. For example, precursor miRNA may have a self-complementary region, which is up to 100% complementary.

Synthetic miRNAs typically comprise two strands, an active strand that is identical in sequence to the mature miRNA that is being studied and a complemenrtary strand that is at least partially complementary to the active strand. The active strand is the biologically relevant molecule and should be preferentially taken up by the complex in cells that modulates translation either through mRNA degradation or translational control. Preferential uptake of the active strand has two profound results: (1) the observed activity of the synthetic miRNA increases dramatically and (2) non-intended effects induced by uptake and activation of the complementary strand are essentially eliminated. According to the invention, several synthetic miRNA designs can be used to ensure the preferential uptake of the active strand.

The introduction of a stable moiety other than phosphate or hydroxyl at the 5′ end of the complementary strand impairs its activity in the miRNA pathway. This ensures that only the active strand of the synthetic miRNA will be used to regulate translation in the cell. 5′ modifications include, but are not limited to, NH2, biotin, an amine group, a lower alkylamine group, an acetyl group, 2 O-Me, DMTO, fluoroscein, a thiol, or acridine or any other group with this type of functionality.

Other sense strand modifications. The introduction of nucleotide modifications like 2′-0Me, NH2, biotin, an amine group, a lower alkylamine group, an acetyl group, DMTO, fluoroscein, a thiol, or acridine or any other group with this type of functionality in the complementary strand of the synthetic miRNA can eliminate the activity of the complementary strand and enhance uptake of the active strand of the miRNA.

As with siRNAs (Schwarz 2003), the relative stability of the 5′ and 3′ ends of the active strand of the synthetic miRNA apparently determines the uptake and activation of the active by the miRNA pathway. Destabilizing the 5′ end of the active strand of the synthetic miRNA by the strategic placement of base mismatches in the 3′ end of the complementary strand of the synthetic miRNA enhances the activity of the active strand and essentially eliminates the activity of the complementary strand.

miR455, microRNA455
or
miR-455 refers to
UAUGUGCCUUUGGACUACAUCG
(miR455-5p; SEQ ID NO: 1)
and/or
GCAGUCCAUGGGCAUAUACAC
(miR455-3p; SEQ ID NO: 2).
miR210, microRNA210
or
miR-210 refers to
AGCCCCUGCCCACCGCACACUG
(miR210-5p; SEQ ID NO: 3)
and/or
CUGUGCGUGUGACAGCGGCUGA
(miR210-3p; SEQ ID NO: 4).

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). In one embodiment, a probe may bind, duplex or hybridise only to the particular target molecule.

The term “stringent conditions” refers to conditions under which a probe will hybridise to its target subsequence, but minimally to other sequences. In some embodiments, a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na+ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Some suitable technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of the subject or control sample, with a plurality of oligonucleotide probes wherein each probe preferentially hybridises to a disclosed gene or genes, or miRNA. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonuleotide arrays may be synthesised in situ (e.g. by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products or miRNAs of approximately the same molecular weight since such similarly sized products are difficult to separate using gels. Accordingly, in such circumstances it may be preferred to compare gene expression using alternative techniques, such as those described below.

Gene expression in a sample representing gene expression and/or levels of miRNAs in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes or miRNAs. Accordingly it will be recognised that such oligonucleotide arrays may be particularly preferred in embodiments where it is desired to compare expression of more than one miRNA and/or gene.

Other suitable methodologies that may be used in the comparison of nucleic acid targets representative of gene expression include, but are not limited to, nucleic acid sequence based amplification (NASBA); or rolling circle DNA amplification (RCA).

It is usually desirable to label probes in order that they may be easily detected. Examples of detectable moieties that may be used in the labelling of probes or targets suitable for use in accordance with the invention include any composition detectable by spectroscopic, photochemical, biochemical, immunochemical, electrical, optical or chemical means. Suitable detectable moieties include various enzymes, prosthetic groups, fluorescent materials, luminescent materials, bioluminescent materials, radioactive materials and colorimetric materials. These detectable moieties are suitable for incorporation in all types of probes or targets that may be used in the methods of the invention unless indicated to the contrary.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, phycoerythrin, texas red, rhodamine, green fluorescent protein, and the like; an example of a luminescent material includes luminol; examples of bioluminescent materials include luciferase, luciferin, and aequorin; examples of suitable radioactive material include ¹²⁵I, ¹³¹I, ³⁵S, ³H, ¹⁴C, or ³²P; examples of suitable colorimetric materials include colloidal gold or coloured glass or plastic (e.g., polystyrene, polypropylene, latex, etc.) beads.

Means of detecting such labels are well known to the skilled person. For example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the coloured label.

In one embodiment of the invention fluorescently labelled probes or targets may be scanned and fluorescence detected using a laser confocal scanner.

In the case of labelled nucleic acid probes or targets suitable labelling may take place before, during, or after hybridisation. In an embodiment, nucleic acid probes or targets are labelled before hybridisation. Fluorescence labels are also suitable and, where used, quantification of the hybridisation of the nucleic acid probes to their nucleic acid targets is by quantification of fluorescence from the hybridised fluorescently labelled nucleic acid. Quantitation may be from a fluorescently labelled reagent that binds a hapten incorporated into the nucleic acid.

In an embodiment of the invention analysis of hybridisation may be achieved using suitable analysis software, such as the Microarray Analysis Suite (Affymetrix Inc.).

Effective quantification may be achieved using a fluorescence microscope which can be equipped with an automated stage to permit automatic scanning of the array, and which can be equipped with a data acquisition system for the automated measurement, recording and subsequent processing of the fluorescence intensity information. Suitable arrangements for such automation are conventional and well known to those skilled in the art.

In an embodiment, the hybridised nucleic acids are detected by detecting one or more detectable moieties attached to the nucleic acids. The detectable moieties may be incorporated by any of a number of means well known to those of skill in the art. However, in an embodiment, such moieties are simultaneously incorporated during an amplification step in the preparation of the sample nucleic acids (probes or targets). Thus, for example, polymerase chain reaction (PCR) using primers or nucleotides labelled with a detectable moiety will provide an amplification product labelled with said moiety. In an alternative embodiment, transcription amplification using a fluorescently labelled nucleotide (e.g. fluorescein-labelled UTP and/or CTP) incorporates the label into the transcribed nucleic acids.

Alternatively, a suitable detectable moiety may be added directly to the original nucleic acid sample (e.g., mRNA, polyA mRNA, cDNA, etc. from the tissue of interest) or to an amplification product after amplification of the original nucleic acid is completed. Means of attaching labels such as fluorescent labels to nucleic acids are well known to those skilled in the art and include, for example nick translation or end-labelling (e.g. with a labeled RNA) by kinasing of the nucleic acid and subsequent attachment (ligation) of a nucleic acid linker joining the sample nucleic acid to a label (such as a suitable fluorophore).

Pre-eclampsia or preeclampsia is a medical condition characterized by high blood pressure and significant amounts of protein in the urine of a pregnant woman. If left untreated, it can develop into eclampsia, the life-threatening occurrence of seizures during pregnancy. There are many different causes for the condition. It appears likely that there are substances from the placenta that can cause endothelial dysfunction in the maternal blood vessels of susceptible women. While blood pressure elevation is the most visible sign of the disease, it involves generalised damage to the maternal endothelium, kidneys, and liver, with the release of vasoconstrictive factors being a consequence of the original damage. An outdated medical term for pre-eclampsia is toxemia of pregnancy, since it was thought that the condition was caused by toxins. Pre-eclampsia may develop at any time after 20 weeks of gestation. Pre-eclampsia before 32 weeks is considered early onset, and is associated with increased morbidity. Its progress differs among patients; most cases are diagnosed before labor typically would begin. Pre-eclampsia may also occur up to six weeks after delivery. Apart from Caesarean section and induction of labor (and therefore delivery of the placenta), there is no known cure. It is the most common of the dangerous pregnancy complications; it may affect both the mother and fetus.

Preeclampsia is diagnosed when a pregnant woman develops both blood pressure >140 systolic and/or >90 diastolic and 0.3 grams or more of protein in a 24-hour urine sample (proteinuria). “Severe pre-eclampsia” involves a BP over 160/110, proteinuria more than 1 g /24 h and signs of end organ damage.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The invention will now be further described with reference to the following Example and figures in which:

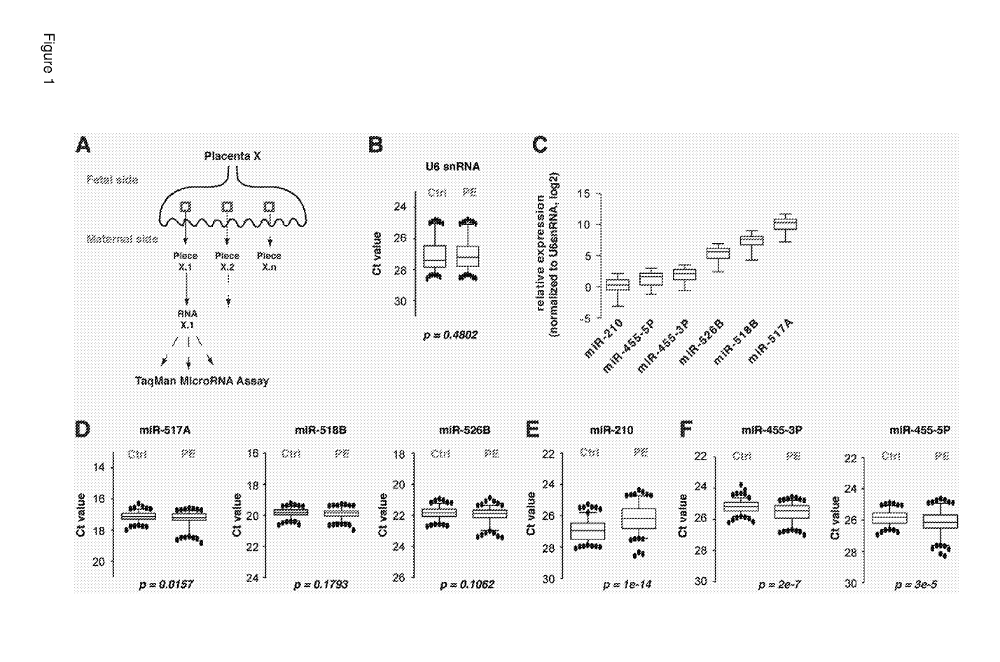

FIG. 1. microRNAs-455 and −210 are deregulated in preeclampsia placentae.

A. Schematic diagram of placenta processing. 14 healthy control and 15 preeclampsia placentae have been collected for the purpose of this study. For each placenta (X), three to four different pieces were dissected from the inner part of the placenta, thereby limiting maternal contamination. Total RNA was extracted from each placenta piece (X.1 to X.n) and microRNA expression was measured in three technical replicates by Taqman qRT-PCR.

B. U6 snRNA expression in PE and Ctrl placentae. U6 level was measured by Taqman qRT-PCR in controls (Ctrl) and preeclampsia (PE) placentae. The cycle threeshold (Ct) value obtained for U6 snRNA was plotted using Whiskers Box Plot 5-95 percentile representation. The p-value was calculated using Mann Whitney test

C. microRNAs 455 are expressed/present in placentae. The expression of three placenta specific (hsa-miR517A, −518B and −526B) and three others microRNAs (hsa-miR210, −455-3P and 455-5P) was analyzed by Taqman qRT-PCR in controls placentae. For each microRNA, their expression was normalized to U6 snRNA level and plotted in log2 scale using Whiskers Box Plot 5-95 percentile representation.

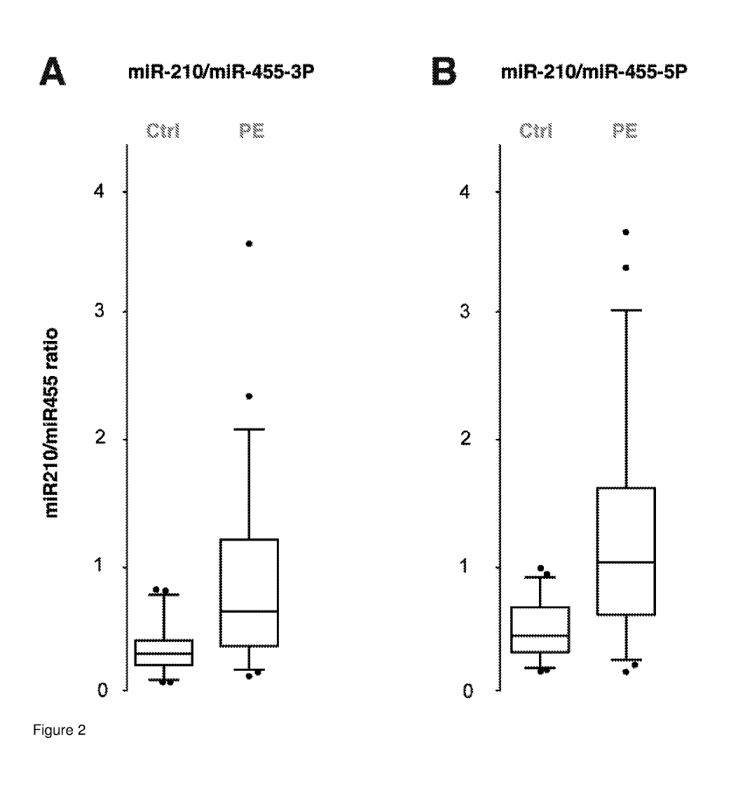

D. to F. As presented in FIG. 2B, the expression of six others microRNAs was evaluated in controls (Ctrl) versus preeclampsia (PE) placentae. The p-value was calculated using Mann Whitney test.

FIG. 2. The miR210/miR455 ratio may serve as a predictive value to diagnose preeclampsia. miRNAs were detected by quantitative real-time RT-PCR using miRNA-specific TaqMan assays. For each placenta, miR210 Ct values were normalized to miR455-3P (left side) or miR-455-5P (right side). Ratios were calculated using DDCt method and were plotted using Whiskers Box Plot 1-99 percentile representation.

### EXAMPLES

### Materials and Methods

Cell Culture and Patient Recruitment

BeWo cells (ACC 458, DSMZ, Germany) were grown at 37° C. in a humidified incubator with 5% CO₂ in Ham's F12 medium supplemented with 20% heat-inactivated Fetal Bovine Serum, Penicillin, Streptomycin and Glutamine (Life Technologies, Switzerland). Forskolin (FSK, 10 uM, 344270) was purchased from Millipore and DMSO from Sigma (D2650).

The prospective case-control study was approved by the local ethical committee. After written informed consent, placenta was collected and processed just after delivery. All patients underwent either elective caesarean section (CS, Controls, n=14) or scheduled CS due to severe preeclampsia (PE, n=15). Severe Preeclampsia was defined as a blood pressure higher than 160/100 mmHg, measured at least 6 hours apart, in combination with proteinuria higher than 2+ (dipstick) at least two times in 24 hours.

3′UTR Cloning and Dual Luciferase Assay

To construct the UTR vectors, a psicheck-2 vector (Promega) containing an Asc1 site was created. Briefly, the psicheck-2 vector was first digested with Not1/Xho1, purified on 1% agarose gel and extracted using Qiaquick Gel extraction Kit (Qiagen). The linearized vector was ligated to the annealed oligonucleotides containing an Asc1 restriction site (Asc1 fwd and Asc1 rev). The vector was digested using Asc1/Not1 enzymes and ciped (except for vectors containing perfect complementary sequences for miR455-3P and -5P).

The 3′UTR were amplified from total RNA extracted from BeWo cells. Briefly, total RNA was reverse transcribed following first strand cDNA synthesis protocol from AffinityScript Multiple temperature cDNA synthesis kit (Agilent) and amplified using iProof High-Fidelity PCR kit (Biorad).

Oligonucleotides were designed to amplify specifically the different UTR using NCBI reference gene and UCSC genome browser (except for the longest HIF1AN 3′UTR that is not amplified/found in BeWo cells but the inventors were able to amplify the shortest UTR from Ensembl genome browser). The amplified UTR were digested using Mlu1/Not1 enzymes.

Digested vector and amplified 3′UTR were ligated using Rapid DNA ligation Kit (Roche Diagnostics). For the control vectors, the oligonucleotides containing the perfect complementary sequences for miR455 3P or 5P were annealed and ligated to unciped digested vector.

BeWo cells were transiently transfected with luciferase reporter constructs following Nanofectin protocol (PAA, Brunschwig). At 48 hours post-transfection, cells were lysed and luciferase activity was measured using Dual Luciferase Reporter assay system (Promega). Renilla luciferase activities (RL) were normalized to Firefly luciferase activity (FL). Measurements were done in technical triplicate and are the results of three independent biological experiments. Datas are presented either as RL/FL ratios or as % repression (ratio RL/FL in FSK condition normalized to the ratio in DMSO condition).

Transient Transfection siRNA and Mimics

siRNA (MUC1, HIF2A, All Stars Negative Control) and synthetic microRNA/mimics (hsa-miR455-3P and −5P) were purchased from Qiagen. Transient siRNA and microRNA mimics transfections in BeWo cells were performed with RNAimax (LifeTechnologies).

RNA Isolation and Expression Analysis

Total RNA with or without microRNAs was extracted from BeWo cells and placenta pieces using the miRVana miRNA isolation kit (LifeTechnologies). The RNA used for pri-miRNA 455 quantification were further treated with Turbo DNA-free kit following the recommendations of the supplier (Life Technologies).

For the placenta, dissection of small pieces (<150 mg) from the villus tree was done within 15 minutes from delivery. After extensive washing in cold PBS, sample was stored 24 hours at 4° C. in RNA later solution (Life Technologies), dried and stored at −80° C. Frozen tissue was directly transferred in pre-chilled lysis solution and homogenized using Polytron PT 2100 (Kinematica AG) and processed as for the cells. The quality of placental RNA samples was estimated using total RNA Chip on Agilent 2100 Bioanalyzer. Only samples with a RIN value superior to 7.5 were considered for further experiments. For mRNA quantification, quantitative qRT-PCR was performed with Taqman One Step RT-PCR Master Mix Reagents kit (LifeTechnologies). To evaluate miRNA and pri-miRNA expression, real time PCR was performed using Taqman MicroRNA reverse transcription kit and High Capacity RNA to cDNA kit respectively, followed by Taqman Universal master mix, no UNG (Life Technologies). The primers used for qPCR experiments were purchased from Life Technologies and are available upon request. All the experiments were performed in triplicate using the StepOne plus real time PCR system for 96 wells plate or the 7900HT Fast real time PCR system for 384 wells plate (Applied Biosystems). All the mRNA and microRNA datas were normalized to RPLP0 and RNU6B respectively, except when mentioned.

Preparation of Small RNA Libraries for High-Throughput Sequencing and Bioinformatic Analysis

The protocol from Emmerth et al. (Dev Cell. 2010 Jan 19;18(1):102-13) was adapted for human small RNA libraries.

After total RNA extraction from BeWo cells using miRVana kit (Ambion), 17-30 nt small RNAs were PAGE purified and were cloned based upon the preactivated, adenylated linkering method described previously (Lau et al.,2001) using a mutant T4 RNA ligase (RnI2 1-249)(Ho et al., 2004). All samples were barcoded at the 3′end of the 5′ adaptor using a hamming distance two code with a 3′cytosine (AAAC, ACCC, AGGC, ATTC, CACC, CCGC,CGTC, CTAC, GAGC, GCTC, GGAC, GTCC, TATC, TCAC, TGCC, TTGC) and sequenced in one lane of an Illumina GAIIx instrument.

Individual reads were assigned to their sample based on the first four nucleotides containing the barcode. The 3′adaptor was removed by aligning it to the read allowing one or two mismatches in prefix alignments of at least seven or ten bases, respectively. Low-complexity reads were filtered out based on their dinucleotide entropy (removing <1% of the reads). All the reads that were shorter than 14 nucleotides were removed. Alignments to the Homo sapiens microRNA database (Human Genome Assembly hg19, mirBase v15) were performed by the software bowtie (Langmead et al., 2009). After assignment of the reads, each microRNA number of reads was normalized to the total number of reads of the library with the lowest number of reads. For the purpose of logarithm scale representation, a number of 2 reads was added to each normalized microRNA number.

Protein Isolation and Western Blot

Total cellular protein was extracted using RIPA buffer (50 mM Tris-HCl pH 7.5, 150 mM NaCl, 1% NP-40, 0.5% sodium deoxycholate, 0.1% SDS, 1 mM EDTA) supplemented with Protease and Phosphatase Inhibitor Cocktail (Roche Applied Science). For the placenta, pieces were dissected as described for RNA preparation but were snap frozen in liquid nitrogen and stored at −80° C. Frozen tissue were thawed for few minutes in pre-chilled RIPA buffer (1 mL per100 mg). Pieces were homogenized with a Polytron (Kinematica) and sonicated 2 times 1 minute (pulse 2 sec on/2 sec off). After centrifugation (13000 rpm, 15 minutes), 20-4 ug of protein were separated on MiniProtean TGX Precast Gel (Biorad) and transferred to a nitrocellulose membrane (Protran, Whatman). The membrane was blocked for 1 hour at room temperature in PBS containing 0.05% Tween-20 and 5% non-fat dry milk, incubated overnight at 4° C. with primary antibodies followed by 1 hour at room temperature with HRP-conjugated secondary antibody. Detection was performed with Western Lightning Plus ECL (Perkin Elmer). The primary antibodies used in this study included antibodies against (((HIF1A (NB 100-105),)))EPAS1/HIF2A (NB 100-122), FIH1/HIF1AN (EPR3658, NBP1-40688), TBP (NB 500-700) and MUC1 (EP1024Y, NB110-57234) from Novus Biologicals. ARNT (ab2771), EGLN2 (ab108980) and MUC1 (ab101352) antibodies were purchased from Abcam.

ImmunoFluorescence in BeWo Cells

Coverslips were cleaned by ethanol: chloric acid (99:1) wash. One coverslip was deposited into one 6 well and sterilized by UV treatment. BeWo cells were plated at the density of 50,000 cells per well. After letting the cells settle for one day, cells were treated with DMSO and FSK for 48 hours. Cells were washed in PBS two times at RT, fixed in 2% PBS-PFA for 5 minutes at RT. The PFA was blocked by adding 0.125 M glycine for 5 minutes. After extensive PBS wash, cells were permeabilized with 0.1% PBS-Triton X-100 for 5 minutes. The slides were transferred to a humid chamber. After 30 minutes of blocking in PBS-BSA (10%), cells were incubated with primary antibody against CDH1 (ab1416, dilution 1/50, Abcam) in PBS-BSA (1%) overnight at 4° C. Cells were washed and incubated with secondary antibody (A-11029, dilution 1/1000, Life Technologies) in PBS-BSA (1%) for 1 hour at RT. After washing, DAPI was applied at the dilution of 1/10000. After extensive wash, the coverslips were mounted on slides with MOWIOL mounting medium. The slides were analyzed using a microscope.

1. A method for predicting a risk for preeclampsia in a subject, said method comprising:
(a) analysing a sample from the subject for the level of expression of miR455 and,
(b) comparing the level of expression of miR455 in the sample from the subject to the levels of miR455 in a control sample, wherein a significantly lower expression of miR455 as compared to the expression of miR455 in the control is indicative of a risk for preeclampsia.

2. The method of claim 1 wherein the expression of miR210 is also analysed in the sample from the subject and wherein the combination of a significantly lower expression of miR455 and a significantly higher expression of miR210, as compared to the control sample, is indicative of a risk for preeclampsia.

3. The method of claim 2, wherein the ratio of the expression of miR210 divided by the expression of miR455 is determined and wherein a ratio greater than 1 is indicative of a risk for preeclampsia.

4. The method of claim 1 wherein the sample comprises placenta tissue.

5. The method of claim 1 wherein the sample is a blood sample.

6. The method of claim 1 wherein the subject is human.

7. A kit for performing the method of comprising:
(i) means for analysing in a sample from a subject the level of expression of miR455; and, optionally,
(ii) means for analysing in a sample from a subject the level of expression of miR210, and, optionally
(iii) means for comparing the level of said miRNAs, i. e. miR455, and, optionally miR210, in a control sample.

8-10. (canceled)
